(19) United States
(12) Patent Application Publication
(10) Pub. No.: US 2022/0096015 A1
(43) Pub. Date: Mar. 31, 2022

(54) MOBILE DEVICE, METHOD OF CONTROLLING THE SAME, AND COMPUTER PROGRAM STORED IN RECORDING MEDIUM

(71) Applicant: SAMSUNG ELECTRONICS CO., LTD., Suwon-si (KR)

(72) Inventors: Andrii OMELCHENKO, Kiev (UA); Kostyantyn SLYUSARENKO, Kiev (UA)

(73) Assignee: SAMSUNG ELECTRONICS CO., LTD., Suwon-si (KR)

(21) Appl. No.: 17/489,204

(22) Filed: Sep. 29, 2021

(30) Foreign Application Priority Data
Sep. 29, 2020 (KR) 10-2020-0126749

(51) Int. Cl. A61B 5/00; A61B 5/0205; A61B 5/024; A61B 5/021; A61B 5/08; A61B 5/1455; A61B 5/16

(52) U.S. Cl. CPC A61B 5/6898; A61B 5/684; A61B 5/6843; A61B 5/742; A61B 5/741; A61B 5/7455; A61B 5/165; A61B 5/0205; A61B 5/02427; A61B 5/021; A61B 5/0816; A61B 5/14552; A61B 5/721

(57) ABSTRACT

A mobile device is provided. The mobile device includes a display, a front camera provided to face in a forward direction of the display, a touch sensor provided at a front side of the display, and a processor configured to acquire a photoplethysmography (PPG) signal from an image of a finger captured by the front camera in a PPG measurement mode, and output guide information that guides at least one of a position of the finger or a contact pressure with which the finger presses the front camera based on at least one of an output of the touch sensor or the image of the finger.

### CROSS-REFERENCE TO RELATED APPLICATION

This application is based on and claims priority to Korean Patent Application No.10-2020-0126749, filed on Sep. 29, 2020 in the Korean Intellectual Property Office, the disclosure of which is incorporated herein by reference.

### BACKGROUND

### 1. Field

The disclosure relates to a mobile device capable of measuring a photoplethysmography (PPG) signal, a method of controlling the same, and a computer program stored in a recording medium.

### 2. Description of the Related Art

A PPG signal is an indicator of changes in blood volume synchronized with a heartbeat, and may be used to acquire not only cardiovascular system related biometric information, such as heart rate, blood oxygenation, arterial blood pressure, stiffness, pulse transition time, pulse wave rate, cardiac output, and arterial compliance, but also other various types of biometric information, such as stress index.

In order to measure a PPG signal, a light source for emitting light of a specific wavelength and a light receiver for receiving light reflected from or transmitted through a human body are required. Recently, mobile devices, such as smartphones and tablet personal computers (PCs) used in daily life are provided with a display that displays an image and a camera that captures an image. .

### SUMMARY

Provided are a mobile device capable of measuring a PPG signal using a display and a camera provided in the mobile device to thereby measure a PPG signal without having additional components or equipment and acquire biometric information based on the PPG signal, and a method of controlling the same.

Further provided are a mobile device capable of providing guide information to a user or correcting distortion based on a position or contact pressure of a finger identified using a touch sensor or a front camera provided in the mobile device to thereby improve the accuracy and reliability of a PPG signal using a basic configuration provided in the mobile device without having additional components or equipment, and a method of controlling the same.

Additional aspects will be set forth in part in the description which follows and, in part, will be apparent from the description, or may be learned by practice of the presented embodiments.

In accordance with an aspect of the disclosure, a mobile device may include a display, a front camera provided to face in a forward direction of the display, a touch sensor provided at a front side of the display, and a processor configured to acquire a PPG signal from an image of a finger captured by the front camera in a PPG measurement mode, and output guide information that guides at least one of a position of the finger or a contact pressure with which the finger presses the front camera based on at least one of an output of the touch sensor or the image of the finger.

In accordance with an aspect of the disclosure, a method of controlling a mobile device that includes a display, a front camera provided to face in a forward direction of the display, and a touch sensor provided at a front side of the display, may include identifying at least one of a position of a finger or a contact pressure with which the finger presses the front camera based on at least one of an output of the touch sensor or an image of the finger captured by the front camera, outputting guide information that guides at least one of the position of the finger or the contact pressure based on a result of the identifying step, and acquiring a PPG signal from the image of the finger captured by the front camera.

### BRIEF DESCRIPTION OF THE DRAWINGS

The above and other aspects, features, and advantages of certain embodiments of the present disclosure will be more apparent from the following description taken in conjunction with the accompanying drawings, in which:

FIGS. 1, 2 and 3 are diagrams illustrating an example of a mobile device according to an embodiment;

FIG. 4 is a diagram illustrating a mobile device according to an embodiment;

FIG. 5 is a diagram illustrating an example of a screen displayed on a display of a mobile device according to an embodiment;

FIG. 6 is a diagram illustrating the position of a user's finger when a mobile device operates in a PPG measurement mode according to an embodiment;

FIG. 7 is a diagram illustrating an example of an emission area of a display when a mobile device operates in a PPG measurement mode according to an embodiment;

FIG. 8 is a diagram illustrating the position of a user's finger when a mobile device operates in a PPG measurement mode according to an embodiment;

FIG. 9 is a diagram illustrating an example of an emission area of a display when a mobile device operates in a PPG measurement mode according to an embodiment;

FIG. 10 is a table showing emission wavelengths according to biometric information, according to an embodiment;

FIG. 11 is a timing diagram illustrating a change in wavelength of light emitted from a display when a PPG signal is measured by emitting light of multi-wavelengths according to an embodiment;

FIGS. 12 and 13 are diagrams illustrating examples of a guide image displayed on a display when a mobile device operates in a PPG measurement mode according to an embodiment;

FIG. 14 is a diagram illustrating a mobile device further including a speaker according to an embodiment;

FIG. 15 is a diagram illustrating an example of a guide speech output through a speaker to guide the position of a finger when a mobile device operates in a PPG measurement mode according to an embodiment;

FIG. 16 shows graphs illustrating a shape of a PPG signal acquired according to a contact pressure of a finger according to an embodiment;

FIG. 17 is a diagram illustrating an example of a guide speech output through a speaker to guide a contact pressure of a finger when a mobile device operates in a PPG measurement mode according to an embodiment;

FIG. 18 is a diagram illustrating a mobile device further including a motion sensor according to an embodiment;

FIG. 19 is a diagram illustrating an example of a warning output in response to a user's hand being moved when a mobile device operates in a PPG measurement mode according to an embodiment;

FIG. 20 is a diagram illustrating an operation performed by a mobile device to correct distortion caused by a change in position of a finger according to an embodiment;

FIGS. 21A, 21B,21C and 21D are graphs showing noise of a PPG signal according to the degree to which a finger moves according to an embodiment;

FIGS. 22 and 23 are diagrams illustrating an operation performed by a mobile device to correct distortion caused by a change in contact pressure of a finger according to an embodiment;

FIG. 24 is a flowchart of a method for controlling a mobile device to provide guide information to a user according to an embodiment;

FIG. 25 is a flowchart of a method of controlling a mobile device to output information for guiding the position of a finger according to an embodiment;

FIG. 26 is a flowchart of a method of controlling a mobile device to output guide information for guiding a contact pressure of a finger according to an embodiment;

FIG. 27 is a flowchart of a method of controlling a mobile device to output guide information with regard to a motion of a finger according to an embodiment;

FIG. 28 is a method of controlling a mobile device to output guide information with regard to a motion of a mobile device according to an embodiment;

FIG. 29 is a flowchart a method of controlling a mobile device to prevent distortion due to a change in position of a finger according to an embodiment; and

FIG. 30 is a flowchart a method of controlling a mobile device to prevent distortion due to a change in contact of a finger according to an embodiment.

### DETAILED DESCRIPTION

Like numerals refer to like elements throughout the specification. Not all elements of embodiments of the present disclosure will be described, and description of what are commonly known in the art or what overlap each other in the embodiments will be omitted. The terms as used throughout the specification, such as “˜ part”, “˜ module”, “˜ member”, “˜ block”, etc., may be implemented in software and/or hardware, and a plurality of “˜ parts”, “˜ modules”, “˜ members”, or “˜ blocks” may be implemented in a single element, or a single “˜ part”, “˜ module”, “˜ member”, or “˜ block” may include a plurality of elements.

It will be further understood that the term “connect” or its derivatives refer both to direct and indirect connection, and the indirect connection includes a connection over a wireless communication network or a connection through an electrical wire.

It should be further understood that the terms “comprises” and/or “comprising,” when used in this specification, identify the presence of stated features, integers, steps, operations, elements, and/or components, but do not preclude the presence or addition of one or more other features, integers, steps, operations, elements, components, and/or groups thereof, unless the context clearly indicates otherwise.

In the specification, it should be understood that, when a member is referred to as being “on/under” another member, it can be directly on/under the other member, or one or more intervening members may in addition be present.

Further, it will be further understood when a signal or data is transferred, sent or transmitted from “an element” to “another element”, it does not exclude another element between the element and the other element passed by the signal or data therethrough, unless the context clearly indicates otherwise.

Although the terms “first,” “second,” “A,” “B,” etc. may be used to describe various components, the terms do not limit the corresponding components, but are used only for the purpose of distinguishing one component from another component. The ordinal numbers used do not indicate the arrangement order, manufacturing order, or importance between components.

As used herein, the singular forms “a,” “an” and “the” are intended to include the plural forms as well, unless the context clearly indicates otherwise.

As used herein, expressions such as “at least one of,” when preceding a list of elements, modify the entire list of elements and do not modify the individual elements of the list. For example, the expression, “at least one of a, b, or c,” should be understood as including only a, only b, only c, both a and b, both a and c, both b and c, or all of a, b, and c.

Reference numerals used for method steps are just used for convenience of explanation, but not to limit an order of the steps. Thus, unless the context clearly dictates otherwise, the written order may be practiced otherwise.

Hereinafter, embodiments of a mobile device, a method of controlling the same, and a computer program stored in a recording medium according to an aspect will be described in detail with reference to the accompanying drawings.

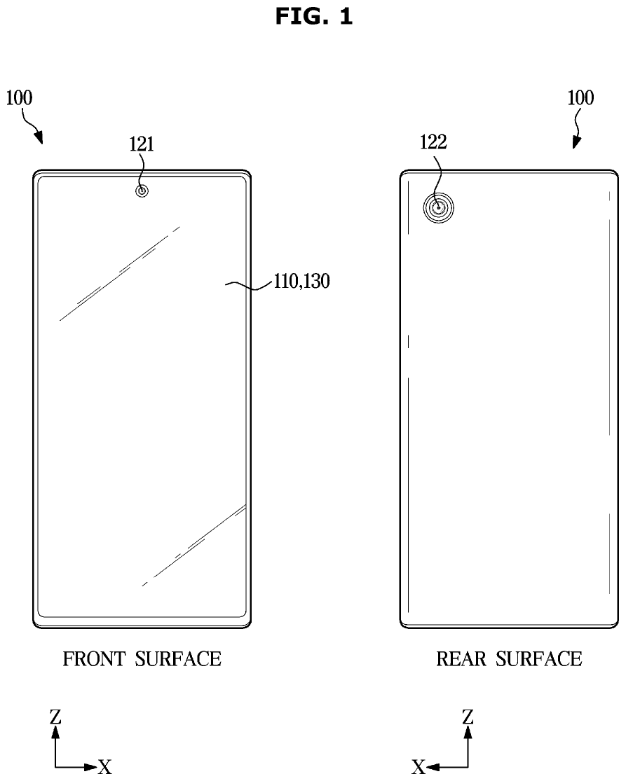

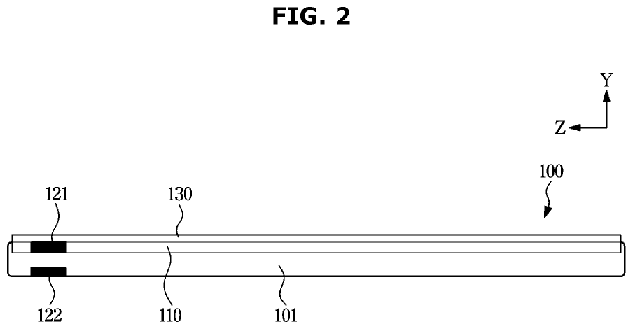

FIGS. 1, 2 and 3 are diagrams illustrating an example of a mobile device according to an embodiment.

A mobile device according to an embodiment may be a portable electronic device having a display and a camera, such as a smart phone or a tablet personal computer (PC). For example, referring to FIG. 1, the mobile device 100 according to the embodiment may include a display 110 for displaying an image and a front camera fc 121 at a front surface thereof and a rear camera 122 at a rear side thereof. However, the rear camera 122 may be omitted according to the design of the mobile device 100.

Referring to the cross-sectional side view of FIG. 2 in conjunction with FIG. 1, a touch sensor 130 may be provided on a front surface of the display 110. Referring to FIG. 2, the touch sensor 130 may be provided in the form of a layer covering almost the entirety of the display 110 to implement a touch screen together with the display 110, and the touch sensor 130 provided in such a form may be referred to as a touch pad or touch panel.

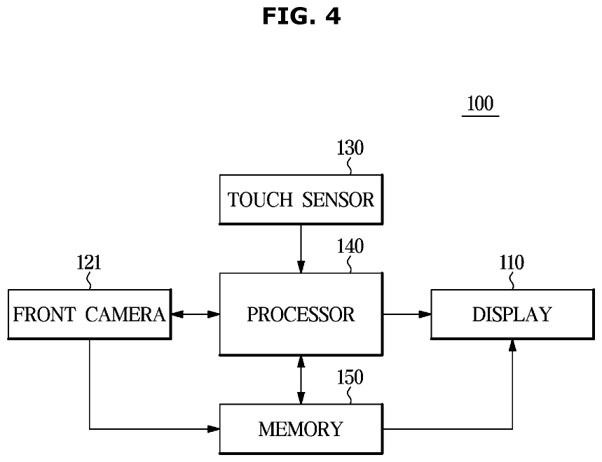

The touch sensor 130 may include an upper plate and a lower plate on which a transparent electrode is deposited, and when information about the position at which a contact has occurred or a change in electrical capacitance has occurred is transmitted to a processor (such as the processor 140 of FIG. 4), the processor may identify a contact position of a user and an input of the user based on the contact based on the transmitted information.

The front camera 121 may be installed into the display 110 and may be located on the rear surface of the touch sensor 130. Referring to FIG. 1, the front camera 121 seen from the front of the mobile device 100 corresponds to a lens of the front camera 121. That is, the front camera 121 may be mounted such that the lens faces in the forward direction of the mobile device 100. Here, the forward direction of the mobile device 100 may refer to a direction (+Y direction) in which the display 110 outputs an image.

Due to the structure of the mobile device 100, when the user touches the lens of the front camera 121, the user is caused to come into contact with the touch sensor 130 provided at the front surface of the front camera 121. Details thereof will be described below.

The rear camera 122 may be mounted in a housing 101 that accommodates and supports the display 110 and other components of the mobile device 100 such that a lens of the rear camera 122 faces in a backward direction of the mobile device 100.

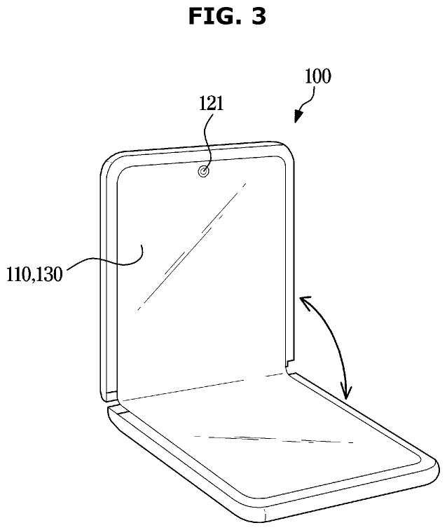

Referring to FIG. 3, the mobile device 100 according to an embodiment may be implemented in a foldable form. The mobile device 100 implemented in a foldable form may be folded such that a part of a front surface of the mobile device is in contact with the other part of the front surface. Alternatively, the mobile device 100 may be provided to be foldable in the opposite direction. Even when the mobile device 100 is implemented in a foldable form, the above descriptions of the positions of the display 110, the touch sensor 130, and the front camera 121 may be applied.

The structure of the mobile device 100 described with reference to FIGS. 1 to 3 is only an example of the mobile device 100 according to an embodiment, and may be variously modified according to a change in design as long as it can perform operations described below.

FIG. 4 is a diagram illustrating a mobile device according to an embodiment.

Referring to FIG. 4, the mobile device 100 according to an embodiment includes a display 110, a front camera 121 provided to face in the forward direction of the display 110, a touch sensor 130 provided at the front side of the display 110, a processor 140 configured to acquire a PPG signal from an image of a finger captured by the front camera 121 in a PPG measurement mode, and a memory 150 in which various pieces of data required for the execution of a program is stored, the program being executed by the processor 140.

The display 110 may employ one of various types of displays, such as a light emitting diode (LED) display, an organic light emitting diode (OLED) display, and a liquid crystal display (LCD).

The display 110 may include a plurality of pixels arranged in two dimensions to implement a two-dimensional image, and each of the pixels may include a plurality of sub-pixels to implement a plurality of colors. For example, in order to implement an RGB image, each of the pixels may include a red sub-pixel, a green sub-pixel, and a blue sub-pixel, and may further include a white sub-pixel or an infrared sub-pixel.

The front camera 121 may include an image sensor, such as a complementary metal oxide semiconductor (CMOS) sensor or a charge coupled device (CCD) sensor. In addition, although not shown in the control block diagram of FIG. 4, when the mobile device 100 includes the rear camera 122, the rear camera 122 may also include an image sensor, such as a CMOS sensor or a CCD sensor.

The touch sensor 130 may be arranged on the front surface of the display 110 in the form of a layer. As a method of the touch sensor 130 detecting a touch, one of various well-known methods, such as a capacitive method, a pressure reduction (e.g., a resistive membrane) method, an ultrasonic method, and an infrared method may be employed.

The mobile device 100 may perform various functions, such as sending/receiving calls and messages, web browsing, and executing various applications. In particular, the mobile device 100 according to an embodiment may perform a PPG measurement function.

The PPG signal is one of the indicators representing changes in blood volume synchronized with the heartbeat. When light of a specific wavelength is transmitted to a human body using a light source, some light is absorbed by blood, bones, and tissues, and some other light is reflected or transmitted and reaches a light receiver. The degree of absorption of light may vary depending on blood, bones, and tissues located in a path through which light passes. Since components except for a change in blood flow caused by a heartbeat are unchanging components, a change in the transmitted light or reflected light received by the light receiver reflects a change in blood volume synchronized with a heartbeat.

The mobile device 100 according to an embodiment may use the display 110 as a light source and the front camera 121 as a light receiver to measure the PPG signal. Accordingly, when the mobile device 100 operates in a PPG measurement mode, the display 110 emits light of a specific wavelength for PPG measurement, and the front camera 121 captures an image of a human body by receiving the light reflected from or transmitted through the human body. That is, the mobile device 100 according to an embodiment may measure the PPG signal using components basically provided in the mobile device 100 without having additional devices, such as additional sensors or light sources.

In the embodiment to be described below, a human body to be subject to PPG measurement will be referred to as a user, and an image captured by receiving light reflected from or transmitted through the human body by the front camera 121 will be referred to as a user image.

Here, the user image only needs to include information (e.g., wavelength information, intensity, etc.) about the light reflected from or transmitted through the user, and does not need to be an image in which the user is identified.

The processor 140 may acquire a PPG signal from the user image captured by the front camera 121. In addition, the processor 140 may acquire biometric information of the user based on the acquired PPG signal, and the biometric information being acquired by the processor 140 may include at least one of a heart rate, a blood oxygenation, a stress index, a respiration rate, a blood pressure, an oxygen delivery time, or a pulse speed. However, the above described biometric information is only an example applicable to the embodiment of the mobile device 100, and it should be understood that various types of biometric information may be acquired by the processor 140.

In addition, the processor 140 may control the overall operation of the mobile device 100. For example, the processor 140 may control the display 110 to emit light of a specific wavelength, and may control the front camera 121 to capture a user image. In the embodiments to be described below, although not mentioned for the sake of convenience of description, it is assumed that operations performed by the display 110, the front camera 121, and other components of the mobile device 100 may be controlled by the processor 140.

A program for executing an operation performed by the processor 140 and various types of data required for executing the program may be stored in the memory 150. A program related to PPG measurement may be stored in the form of an application, and such an application may be installed by default in the mobile device 100 or may be installed by a user after the mobile device 100 is sold.

In the latter case, the user may install the application for PPG measurement in the mobile device 100 by downloading the application for PPG measurement from a server providing the application.

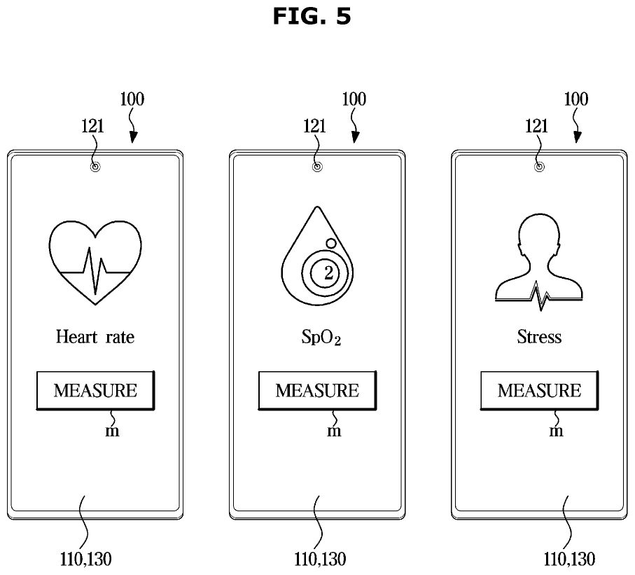

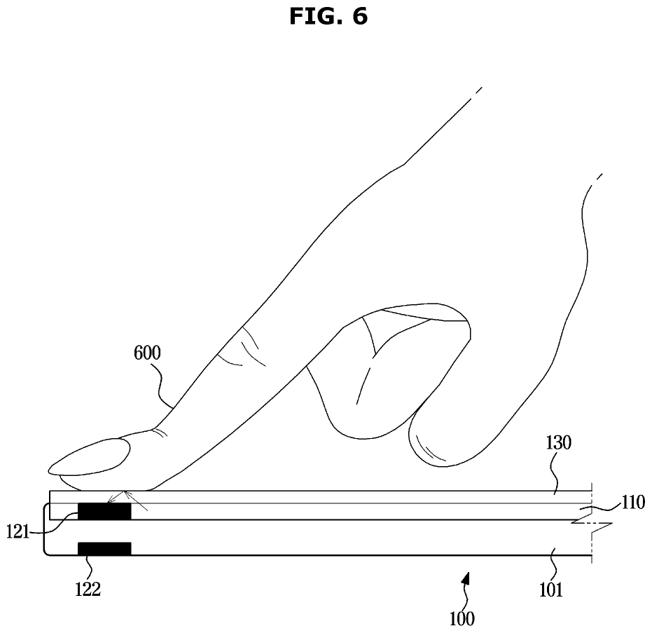

FIG. 5 is a diagram illustrating an example of a screen displayed on a display of a mobile device according to an embodiment.

When an application for PPG measurement is executed in the mobile device 100 according to an embodiment, the mobile device 100 may operate in a PPG measurement mode. As described above, the mobile device 100 operating in the PPG measurement mode may measure at least one type of biometric information among a heart rate, a blood oxygenation (SpO2), a stress index, a respiration rate, a blood pressure, an oxygen delivery time and a pulse speed. The mobile device 100 may provide a result of the measurement to the user.

For example, when an application for PPG measurement is executed in the mobile device 100, a screen for selecting biometric information desired to be measured may be displayed on the display 110 as shown in FIG. 5. As in the example of FIG. 5, one pieces of measurable biometric information may be displayed on one screen displayed on the display 110, and a user who desires to measure the displayed biometric information may select the displayed biometric information by touching a measurement button m displayed on the screen.

When the biometric information displayed on the screen is not biometric information desired to be measured, the user may swipe the screen to move to the next screen, and when a screen of desired biometric information is displayed, the user may touch the measurement button m.

Alternatively, a plurality of measurement buttons m respectively corresponding to a plurality of pieces of measurable biometric information may be displayed on one screen.

When biometric information is selected by the user, the mobile device 100 may perform a series of operations for measuring the selected biometric information. Hereinafter, the operations will be described in detail.

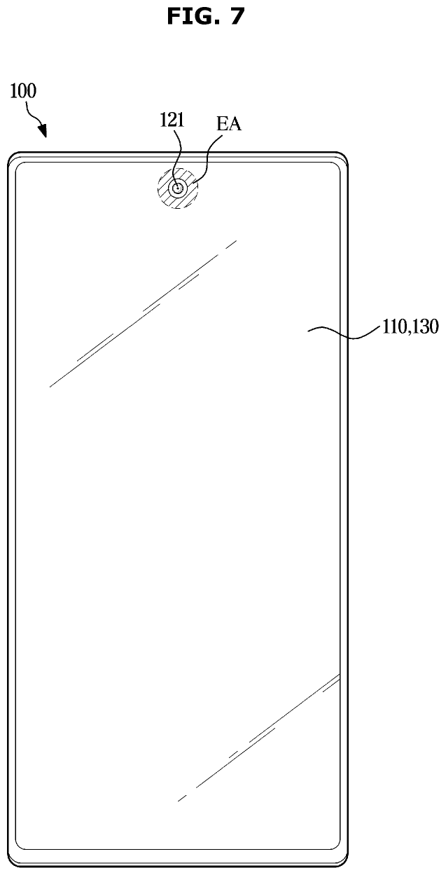

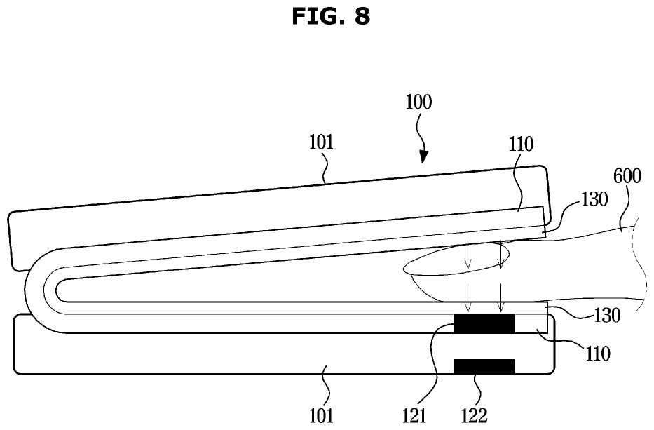

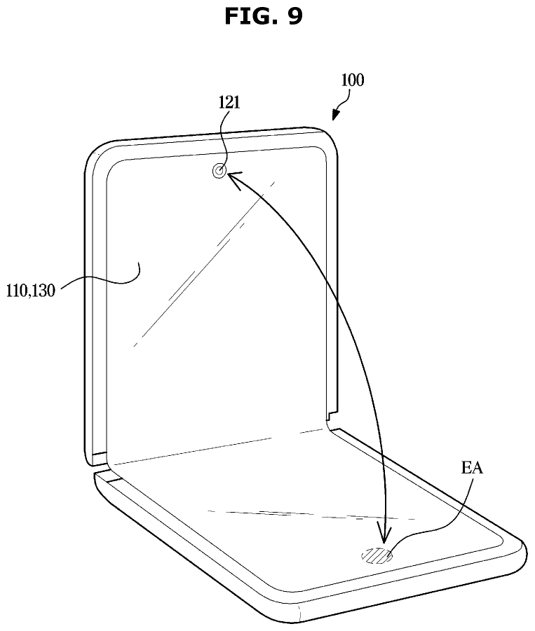

FIG. 6 is a diagram illustrating the position of a user's finger when a mobile device operates in a PPG measurement mode according to an embodiment. FIG. 7 is a diagram illustrating an example of an emission area of a display when a mobile device operates in a PPG measurement mode according to an embodiment. FIG. 8 is a diagram illustrating the position of a user's finger when a mobile device operates in a PPG measurement mode according to an embodiment. FIG. 9 is a diagram illustrating an example of an emission area of a display when a mobile device operates in a PPG measurement mode according to an embodiment.

The pressure generated by the heartbeat allows blood to flow in blood vessels, and the pressure by the heartbeat acts up to the end capillaries of the human body. Arterial blood from the capillaries of the fingertips supplies blood to the tissues, enters the veins, and returns to the heart. Accordingly, the arterial blood volume in the fingertip capillaries repeatedly increases and decreases in synchronization with the heartbeat.

As described above, the PPG signal is an index indicating a change in blood volume synchronized with a heartbeat. Therefore, the measurement of the PPG signal may be performed at the extremities of the body, such as a finger, toe, or earlobe. For the sake of convenience of measurement, the following description will be made in relation to a case in which a PPG signal is measured on a finger of a user as an example.

Referring to FIG. 6, when the finger 600 of the user is positioned on the front surface of the front camera 121 serving as a light receiver (e.g., on the front surface of the lens of the front camera 121), light of a specific wavelength may be emitted from an area corresponding to the front camera 121 of the display 110.

When the PPG signal is measured in a reflective type, light of a specific wavelength may be emitted from an area adjacent to the front camera 121. For example, as shown in FIG. 7, light of a specific wavelength may be emitted from a circular area having a predetermined diameter with respect to the center of the lens of the front camera 121. In the embodiment to be described below, an area of the display in which light is emitted for measuring the PPG signal is referred to as an emission area EA. However, the shape of the emission area EA is not limited to a circular shape, and may be implemented in a polygonal shape, such as a quadrangle or a hexagon, or other shapes.

According to the design of the mobile device 100, components of the display 110 emitting light may be disposed on the front surface of the lens of the front camera 121, or components of the display 110 emitting light may not be disposed on the front surface of the lens of the front camera 121. In the former case, light may be emitted from the front surface of the lens of the front camera 121, and in the latter case, light may not be emitted from the front surface of the lens of the front camera 121 (i.e., the shape of the emission area EA may be a shape in which the center is empty).

In addition, the touch sensor 130 may or may not be located on the front surface of the lens of the front camera 121. Because the user's finger 600 is larger than the lens of the front camera 121, when the user places the finger 600 on the lens of the front camera 121, the finger 600 is caused to come into contact with the touch sensor 130 around the lens regardless of whether the touch sensor 130 is located on the front surface of the lens of the front camera 121.

Referring again to FIG. 6, light emitted from the emission area EA of the display 110 reaches the user's finger 600. Some of the light reaching the finger 600 is absorbed by bones, blood, tissue, etc., and other some of the light is reflected and then incident onto the lens of the camera 121.

The front camera 121 may capture the finger image by receiving the reflected light incident onto the lens, and the processor 140 may acquire a PPG signal from the captured finger image.

Referring to FIG. 8, when the mobile device 100 is implemented in a foldable form, the user's finger 600 may be placed on the front surface of the lens of the front camera 121 and the mobile device 100 may be folded. In this case, light of a specific wavelength may be emitted from an area of the display 110 corresponding to the front camera 121. Here, the area corresponding to the front camera 121 may represent an area facing the front camera 121 when the mobile device 100 is folded.

That is, when the mobile device 100 is implemented in a foldable form, light of a specific wavelength is emitted from the opposite side rather than from the same side as the front camera 121 such that the front camera 121 may receive the light transmitting through the finger.

For example, referring to FIG. 9, an emission area EA having a predetermined size and a predetermined shape may be formed in an area of the display 110 corresponding to the front camera 121. Although the emission area EA is illustrated as a circular shape in the example of FIG. 9, the embodiment of the mobile device 100 is not limited thereto, and the emission area EA may be implemented as a polygonal shape such as a square or hexagon, or other shapes.

However, even when the mobile device 100 is implemented in a foldable form, the emission area EA may be formed in an area adjacent to the front camera 121 as shown in FIG. 7, such that the front camera 121 receives light reflected from the finger.

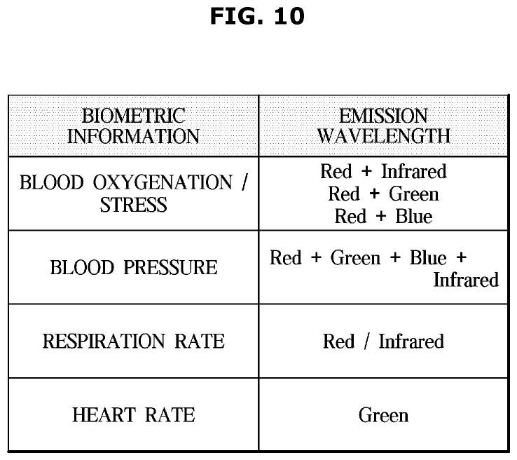

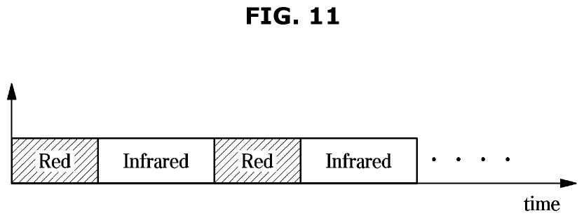

FIG. 10 is a table showing emission wavelengths according to biometric information, according to an embodiment. FIG. 11 is a timing diagram illustrating a change in wavelength of light emitted from a display when a PPG signal is measured by emitting light of multi-wavelengths according to an embodiment..

The wavelength of light emitted from the emission area EA may vary depending on biometric information to be measured. For example, as shown in FIG. 10, when it is desired to measure a blood oxygenation or stress index, a combination of red light and infrared light, a combination of red light and green light, or a combination of red light and blue light may be emitted from the emission area EA.

When it is desired to measure a blood pressure, a combination of red light, green light, blue light, and infrared light may be emitted from the emission area EA, and when it is desired to measure a respiration rate, red light or infrared light may be emitted, and when it is desired to measure a hear rate, green light may be emitted.

However, the table of FIG. 10 is only an example applicable to the mobile device 100 and the embodiment of the mobile device 100 is not limited thereto. While red light responds most sensitively to changes in blood volume, hemoglobin in blood exhibits the highest absorption in a green wavelength band. In addition, in general, noise appearing in green light and blue light is less than that appearing in red light. As described above, since the advantages and disadvantages of each wavelength band are different, it will be understood that biometric information and emission wavelengths may be matched differently from the table of FIG. 10 in consideration of the advantages and disadvantages of each wavelength.

Information about the emission wavelengths each matched with corresponding biometric information may be stored in the memory 150, and when biometric information is selected by a user, the processor 140 may control the display 110 to emit light of an emission wavelength matched with the selected biometric information from the emission area EA.

The depth transmitted through the human tissue may vary depending on the wavelength band. Accordingly, when the PPG signal is measured using multi-wavelengths, more diverse and accurate information may be acquired. Referring to the example of FIG. 10, emission wavelengths matched with a blood oxygenation, a stress index, a blood pressure, and a respiration rate correspond to multi-wavelengths.

As described above, the display 110 of the mobile device 100, which includes a plurality of sub-pixels for each single pixel, may implement various colors, and thus may emit light of various wavelengths for acquiring biometric information.

In order to measure the PPG signal using multi-wavelength light, the processor 140 may control the display 110 to emit light from at least two of the red sub-pixel, the green sub-pixel, or the blue sub-pixel included in the emission area EA. In some cases, an infrared sub-pixel may also be used.

For example, when using red light and infrared light to measure the respiration rate, as shown in FIG. 11, red light and infrared light may be alternately emitted from the emission area EA. Even when three or more multi-wavelengths are used, each light may be alternately emitted, and there is no restriction on the emission order or emission time. Alternatively, light rays of multiple wavelengths may be simultaneously emitted.

Light emission from the emission area EA may be performed after biometric information is selected. The light emission may be performed immediately after selection of biometric information, or may be performed after the user's finger 600 contacts the lens of the front camera 121, or may be performed when it is confirmed that the user's finger is properly positioned.

Light emitted from the emission area EA of the display 110 may be reflected from or transmitted through a finger 600 and then be incident onto the lens of the front camera 121. The front camera 121 may capture frame images according to a set frame rate, and each of the frame images captured by receiving light reflected from or transmitted through a finger 600 may be referred to as a finger image.

The finger image captured by the front camera 121 may be transmitted to the processor 140, and the processor 140 may acquire a PPG signal from the finger image. In addition, the processor 140 may identify or calculate the biometric information selected by the user using the acquired PPG signal.

For example, the processor 140 may extract a specific wavelength component from the finger images captured at regular time intervals. A change in a value of the specific wavelength component according to time change may indicate a PPG signal. The processor 140 may divide a specific wavelength component into an alternating current (AC) component and a direct current (DC) component, and calculate the selected biometric information using the divided AC component and DC component.

The calculated biometric information may be provided to the user through the display 110 or the speaker 160, and may be used to provide healthcare-related services. For example, the calculated biometric information may be used to monitor a health status of a user having a specific disease. When the calculated biometric information is out of a reference range, a warning message may be output or relevant information may be transmitted to a related medical institution.

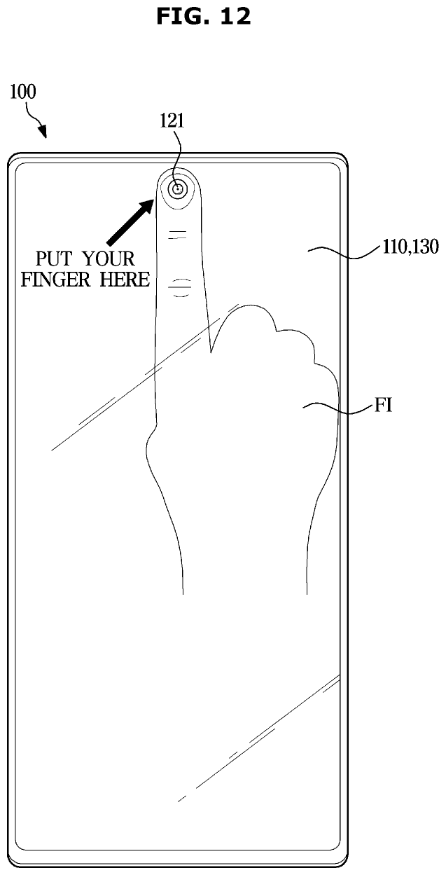

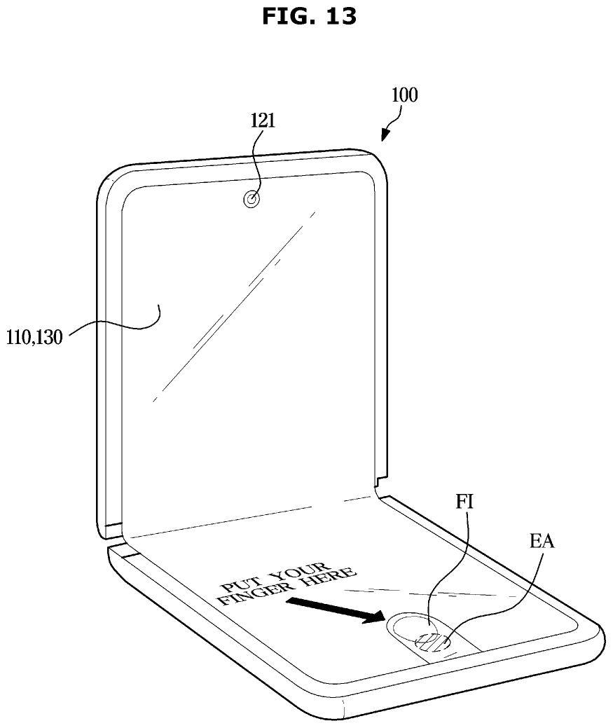

FIGS. 12 and 13 are diagrams illustrating examples of a guide image displayed on a display when a mobile device operates in a PPG measurement mode according to an embodiment.

For accurate measurement of the PPG signal, it is important that the user's finger 1200 is placed at an accurate position corresponding to the front camera 121. Accordingly, the mobile device 100 according to an embodiment may output guide information for guiding the position of the finger. For example, guide information for guiding the position of a finger may be output using at least one of a visual method, an auditory method, or a tactile method.

For example, as shown in FIG. 12, a position guide image for guiding the position of a finger may be displayed on the display 110. The position guide image may include visual content for guiding the user's fingertip to be positioned on the front surface of the lens of the front camera 121.

For example, the position guide image may include an arrow pointing to the lens of the front camera 121 or a finger-shaped image FI. When the finger-shaped image FI is displayed on the display 110, the user may place his or her finger to overlap the finger-shaped image Fl displayed on the display 110.

As another example, when the mobile device 100 is implemented in a foldable form, as shown in FIG. 13, a finger may be placed on the opposite side such that the mobile device 100 may be folded. To this end, the position guide image may also be displayed upside down to guide the user's hand to be positioned on the upper side of the mobile device 100.

The embodiment of the mobile device 100 is not limited to the examples of FIGS. 12 and 13 described above. As another example, when the mobile device 100 is not implemented in a foldable form, the user's hand may be guided to be positioned on the upper side of the mobile device 100. Alternatively, when the mobile device 100 is implemented in a foldable form, the user's hand may be guided to be positioned as shown in FIG. 12 and the PPG signal may be measured in a state in which the mobile device 100 is not folded.

The mobile device 100 according to an embodiment may output guide information for guiding at least one of a finger position or a finger contact pressure, or perform a distortion preventive process for preventing distortion due to motion of the finger based on an output of the touch sensor 130 and an output of the front camera 121. The guide information output in this case may also be output using at least one of a visual method, an auditory method, or a tactile method. Hereinafter, detailed operations thereof will be described.

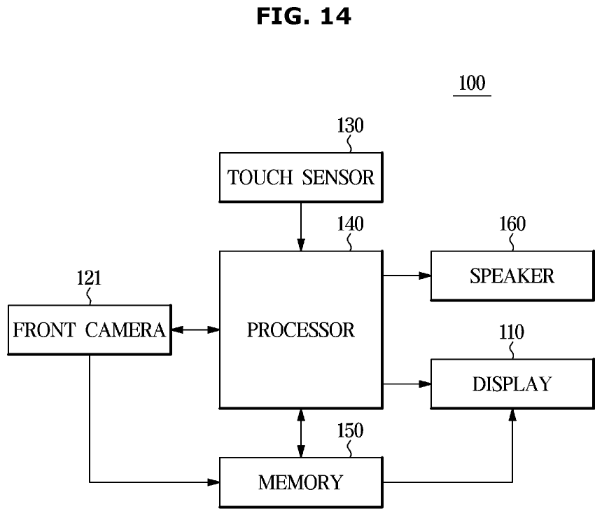

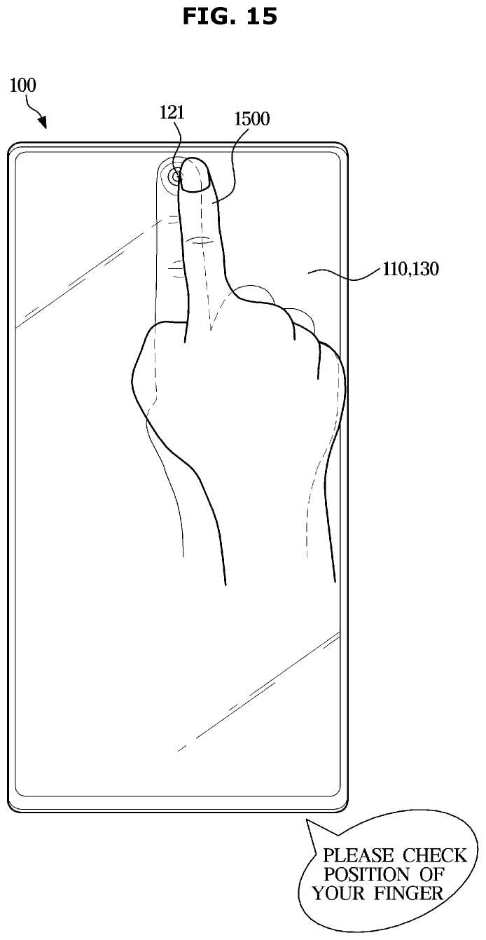

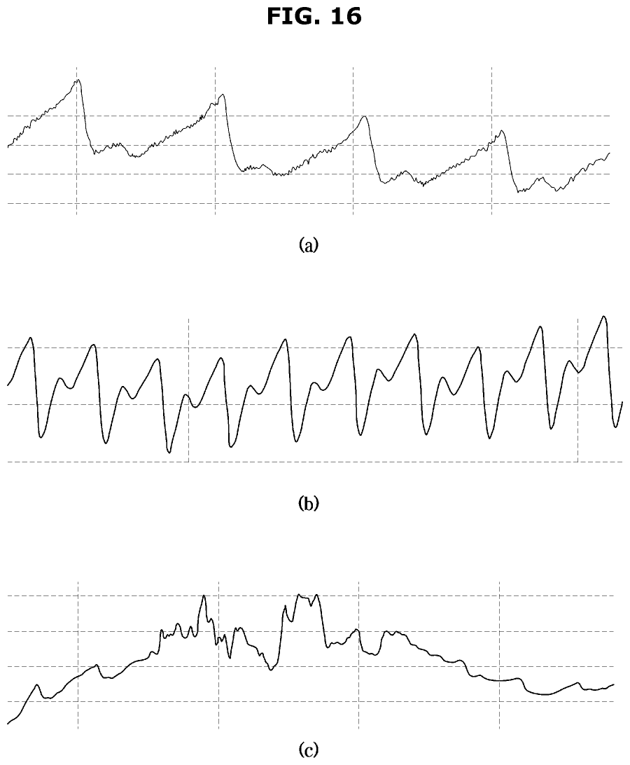

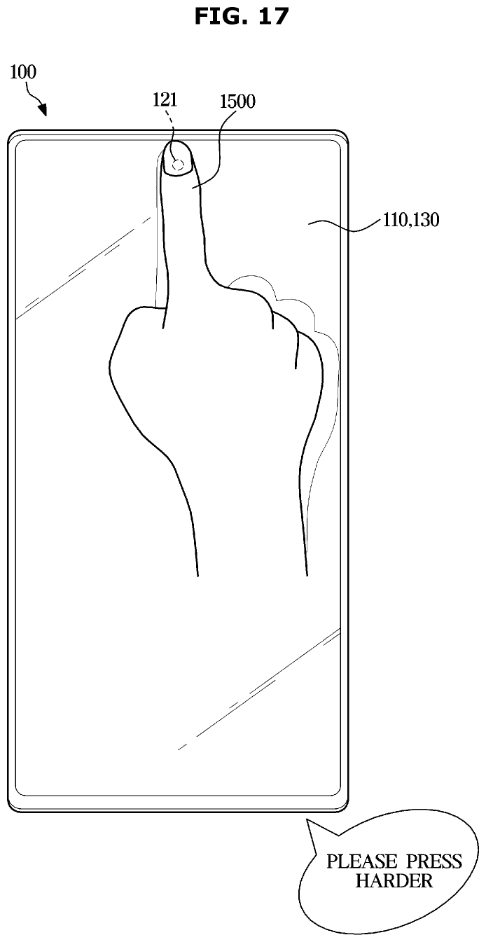

FIG. 14 is a diagram illustrating a mobile device further including a speaker according to an embodiment. FIG. 15 is a diagram illustrating an example of a guide speech output through a speaker to guide the position of a finger when a mobile device operates in a PPG measurement mode according to an embodiment. FIG. 16 shows graphs illustrating a shape of a PPG signal acquired according to a contact pressure of a finger according to an embodiment. FIG. 17 is a diagram illustrating an example of a guide speech output through a speaker to guide a contact pressure of a finger when a mobile device operates in a PPG measurement mode according to an embodiment.

Referring to FIG. 14, the mobile device 100 according to an embodiment may further include a speaker 160 that outputs a guide speech for measuring an accurate PPG signal. The speaker 160 may be provided in at least one area of the housing 101 of the mobile device 100.

The guide speech output through the speaker 160 may include information for guiding the position of the finger 1500 or the contact pressure. The contact pressure may refer to a pressure of the user's finger 1500 with which the lens of the front camera 121 is pressed.

When the above-described guide image is displayed on the display 110, a guide speech corresponding to the displayed guide image (e.g., a guide speech, such as “place your finger on the lens of the front camera”) may be output through the speaker 160 together with the guide image.

In addition, when the user's finger 1500 is not placed according to a predetermined position or a predetermined pressure, a guide speech for correcting the position of the finger 1500 or the contact pressure may be output.

The processor 140 may identify whether the user's finger 1500 is located in a predetermined area on the display 110 based on the output of the touch sensor 130, and output the identification result using at least one of a visual, auditory, or tactile manner. In the present example, a case of outputting using an auditory method will be described.

The predetermined area may be an area in which a finger 1500 needs to be positioned for PPG signal measurement, and has a predetermined size or a predetermined shape at a predetermined position. For example, the predetermined area may be defined as a circular or rectangular area having a predetermined size with respect to the center of the front camera 121.

The output of the touch sensor 130 indicates the position of the touch sensor 130 being in contact with an object. Accordingly, the processor 140 may identify the position of the finger 1500 being in contact with the touch sensor 130 based on an output of the touch sensor 130. Alternatively, the processor 140 may identify the position of the finger 1500 in contact with the touch sensor 130 based on an output of the front camera 121 (i.e., a finger image captured by the front camera 121). In particular, when the resolution of the front camera 121 is higher than the resolution of the touch sensor 130, the accuracy of position identification may be improved using the output of the front camera 121.

Information about the above-described predetermined area may be stored in the memory 150, and the processor 140 may compare the finger position, for which the output of the touch sensor 130 is provided, with the information about the predetermined area stored in the memory 150 to identify whether the finger 1500 is located in the predetermined area.

Referring to FIG. 15, when it is identified that the finger 1500 is not located in the predetermined area, the processor 140 may control the speaker 160 to output a guide speech, such as “please check the position of the finger”. Alternatively, in order to provide more detailed guide information, a guide speech, such as “move your finger to the left” may be output.

Alternatively, guide information may be provided in a visual manner by outputting information, which is output as a guide speech, in the form of text, and upon identifying that the finger 1500 is not located in the predetermined area, vibration may be generated in the mobile device 100 to provide guide information in a tactile method.

On the other hand, when the finger 1500 comes in contact with the lens of the front camera 121 serving as a light receiver with a suitable pressure, a more accurate PPG signal may be measured. When the pressure of the finger 1500 pressing the lens of the front camera 121 is weak, a PPG signal having a shape as shown in (a) of FIG. 16 is acquired, and the pressure of the finger 1500 pressing the lens of the front camera 121 is suitable (within a predetermined range), a PPG signal having a shape as shown in (b) of FIG. 16 may be acquired. In addition, when the pressure of the finger 1500 pressing the lens of the front camera 121 is high, a PPG signal having a shape as shown in (c) of FIG. 16 may be acquired.

Among the three PPG signals shown in FIG. 16, the most suitable type of PPG signal for acquiring biometric information is the PPG signal shown in (b) of FIG. 16. Accordingly, the pressure applied at a time when acquiring the PPG signal shown in (b) of FIG. 16 may be identified as a suitable pressure, and the pressure may be set and stored through a test performed at the first execution of the PPG measurement mode. Alternatively, the suitable pressure may also be set and stored in advance by experiments, simulations, theories, statistics, etc. in the manufacturing stage of the mobile device 100.

The processor 140 may determine the contact pressure of the finger based on at least one of the output of the touch sensor 130 or the output of the front camera 121, and may output guide information for guiding the finger contact pressure to fall within a predetermined range using at least one of a visual manner, an auditory manner, or a tactile manner.

When the touch sensor 130 is implemented in a resistive membrane method and thus is capable of directly measuring the finger contact pressure, the processor 140 may directly identify the finger contact pressure based on the output of the touch sensor 130.

When the touch sensor 130 is implemented in a non-resistive membrane method, and thus is incapable of directly measuring the contact pressure of the finger, the processor 140 may indirectly identify the contact pressure of the finger 1500 based on the contact area between the finger 1500 and the touch sensor 130. For example, the contact pressure may be identified to be greater as the contact area between the finger 1500 and the touch sensor 130 is larger, and weaker as the contact area is smaller.

The contact area between the finger 1500 and the touch sensor 130 may be identified based on the output of the touch sensor 130, or may be identified based on the output of the front camera 121 (i.e., the finger image captured by the front camera 121).

As a result of the determination, when it is identified that the contact pressure of the finger 1500 is weaker than a predetermined range of pressures as shown in FIG. 17, the processor 140 may control the speaker 160 to output a guide speech, such as “please press harder”. Conversely, when it is identified that the contact pressure of the finger 1500 is greater than the predetermined pressure, the processor 140 may control the speaker 160 to output a guide speech, such as “please press weaker”.

Alternatively, text having the same content as that of the guide speech may be displayed on the display 110 to output guide information in a visual manner, or vibration may be generated in the mobile device 100 to output guide information in a tactile manner.

For example, the mobile device 100 may guide the contact pressure of the finger 1500 after guiding the position of the finger first. That is, as described above, the processor 140 may identify the position of the user's finger 1500 based on the output of the touch sensor 130 or the output of the front camera 121. When the position of the finger 1500 is not located in a predetermined area, the processor 140 may output information for guiding the position to the predetermined area, and then when a result of re-identification is that the position of the user's finger 1500 is located in the predetermined area, the processor 140 may identify the finger contact pressure, and output information for guiding the finger's contact pressure according to the identification result.

As another example, the contact pressure of the finger 1500 may be guided first, or the position of the finger and the contact pressure of the finger may be simultaneously guided.

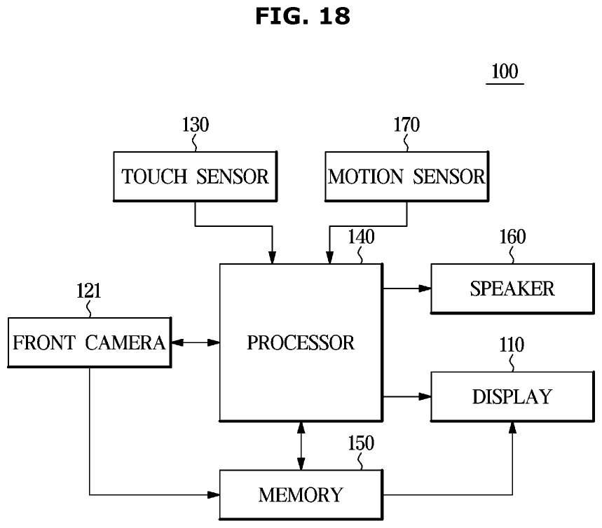

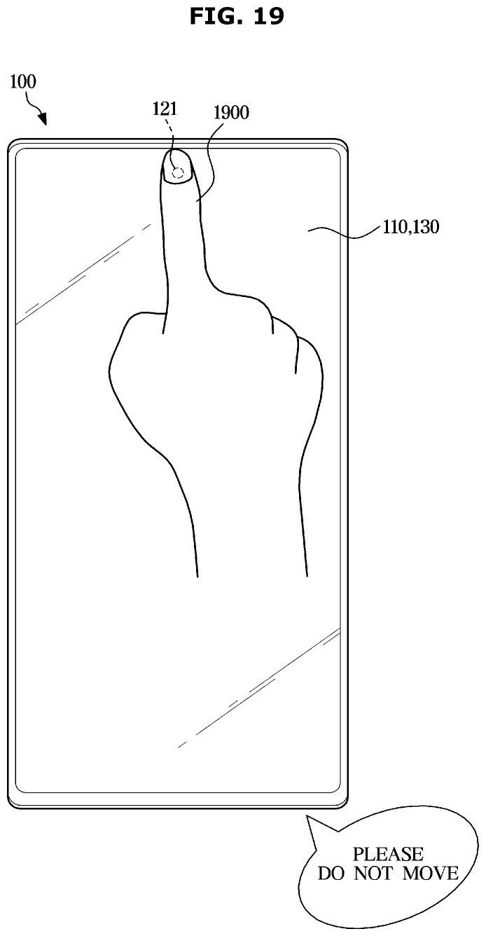

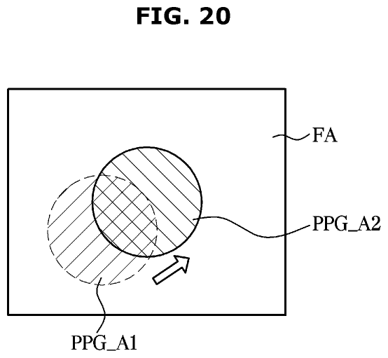

FIG. 18 is a diagram illustrating a mobile device further including a motion sensor according to an embodiment. FIG. 19 is a diagram illustrating an example of a warning output in response to a user's hand being moved when a mobile device operates in a PPG measurement mode according to an embodiment.

Referring to FIG. 18, the mobile device 100 according to an embodiment may further include a motion sensor 170 for detecting a motion of the mobile device 100. For example, the motion sensor 170 may include at least one of an acceleration sensor or a gyro sensor.

The processor 140 may determine whether the mobile device 100 moves based on the output of the motion sensor 170, and may output guide information related to the motion of the mobile device 100.

When the processor 140 identifies that the mobile device 100 has moved in the PPG measurement mode, the processor 140 may output guide information for indicating that a motion of the mobile device 100 is not allowed, such that distortion of the PPG signal due to the motion of the mobile device 100 is prevented. In the example of FIG. 19, the guide information is illustrated as being output in an auditory manner, but the guide information may be output in a visual or tactile manner, or may be output in a combination of two or more methods.

The processor 140 may identify the motion of the user's finger 1900 based on the output of the touch sensor 130 or the output of the front camera 121. The motion of the finger 1900 may include at least one of a change in position of the finger 1900 or a change in a contact pressure of the finger 1900. Accordingly, the processor 140 may identify the change in position of the finger 1900 or the change in contact pressure of the finger 1900 based on the output of the touch sensor 130 or the output of the front camera 121.

When the processor 140 identifies that the user's finger 1900 has moved based on the output of the touch sensor 130 or the output of the front camera 121, the processor 140 may output guide information for indicating that a motion is not allowed using at least one of a visual method, an auditory method, or a tactile method similar to the above.

On the other hand, when the motion of the finger 1900 occurs in response to the guide information for preventing the motion of the finger 1900 being output during a PPG measurement, the processor 140 may perform a distortion preventive process to prevent distortion due to the motion of the finger 1900 using various components provided in the mobile device 100. The distortion caused by the motion of the finger 1900 may include at least one of noise or artifacts appearing in the PPG signal.

For example, when a finger motion has occurred in response to the guide information being output a predetermined number of times, the processor 140 may perform the distortion preventive process, which will be described below.

Alternatively, the output of the guide information for the motion may be omitted, and the distortion preventive process, which will be described below, may be performed.

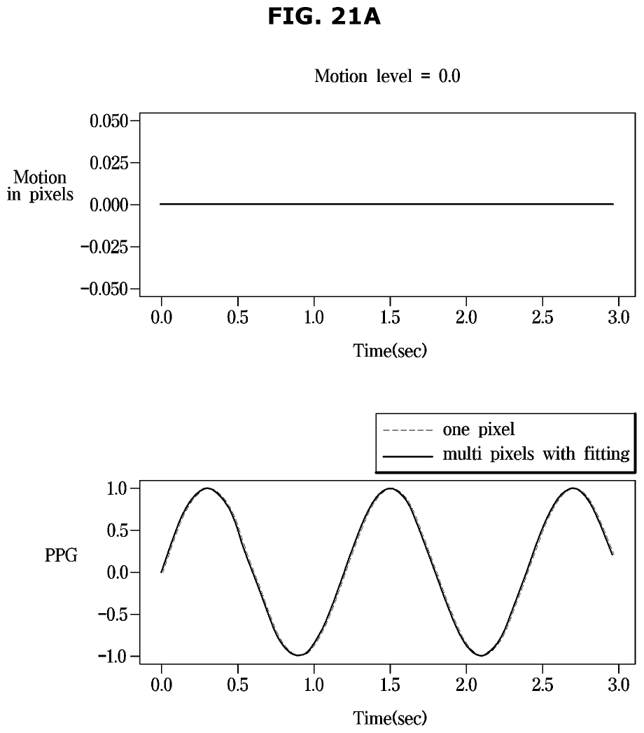

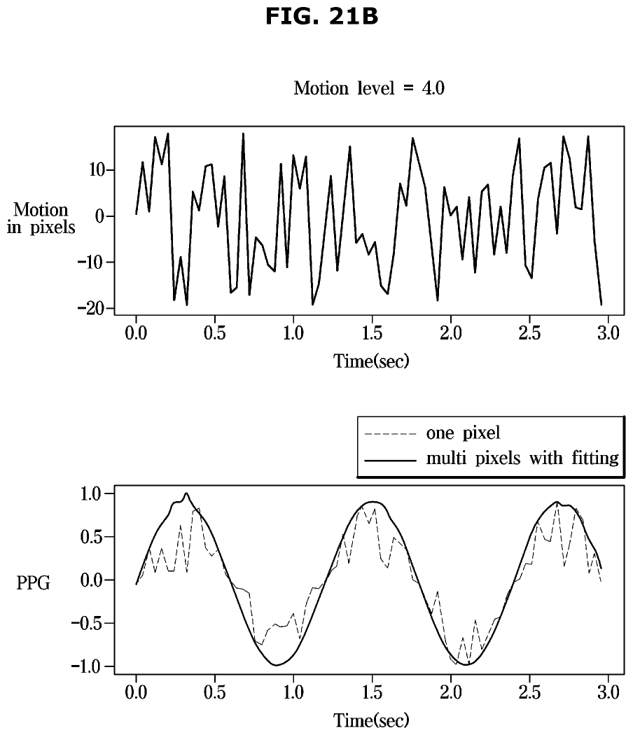

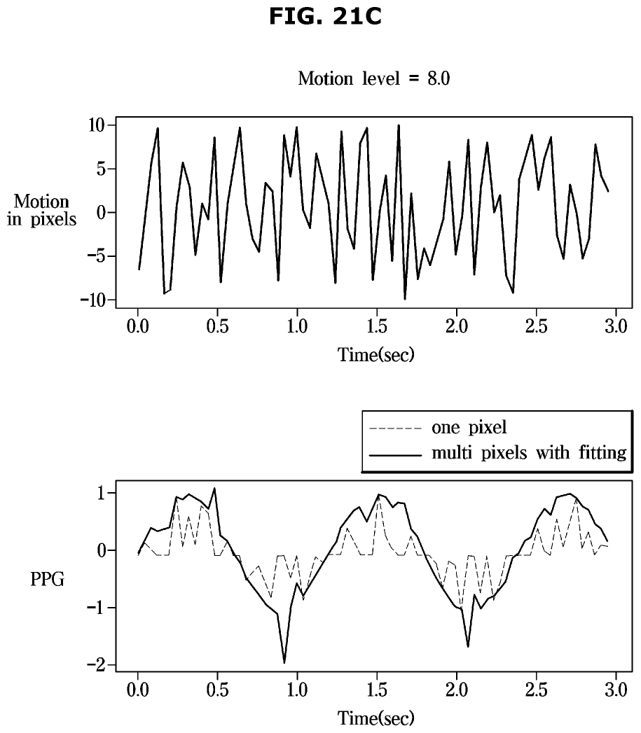

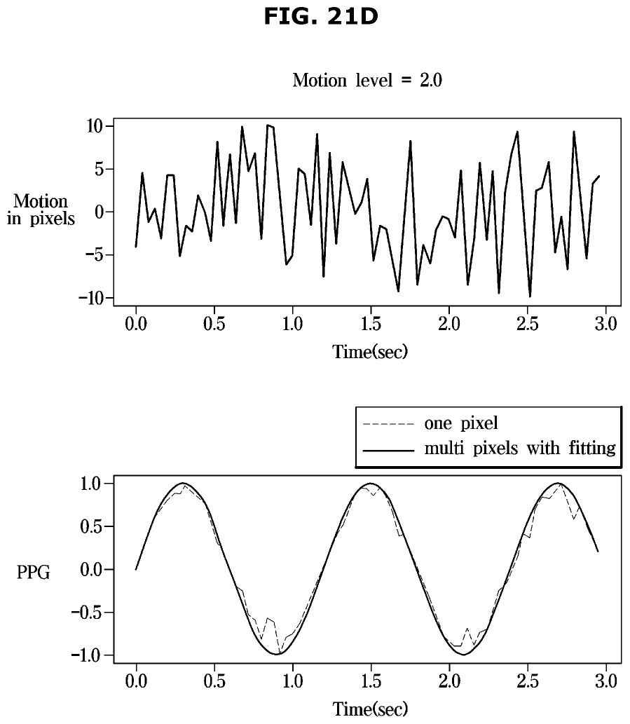

.20 is a diagram illustrating an operation performed by a mobile device to correct distortion caused by a change in position of a finger according to an embodiment. FIGS. 21A, 21B, 21C and 21D are graphs showing noise of a PPG signal according to the degree to which a finger moves according to an embodiment.

When assuming a frame area FA of the front camera 121 shown in FIG. 20, the following description is made in relation that an area in contact with the user's finger in the frame area FA for measuring the PPG signal is shifted from a first area PPG_A1 to a second area PPG_A2 due to the motion of the finger.

In the example, the frame area FA of the front camera 121 refers to an area included in a frame image captured by the front camera 121 (i.e., a coverage of the front camera 121). The frame area FA may be an area set assuming a case in which the user's finger is in contact with the lens of the front camera 121.

In order to prevent distortion due to the motion of the finger, the processor 140 may track the motion of the finger based on the output of the touch sensor 130 or the output of the front camera 121, and determine at least one pixel to be used for acquiring a PPG signal from a finger image based on the current position of the finger.

As described above, a plurality of frame images captured by the front camera 121 may be used to acquire the PPG signal, and the plurality of frame images may be captured according to a set frame rate and transmitted to the processor 140.

The processor 140 may extract the PPG signal from at least one pixel corresponding to the current position of the finger in the transmitted frame image. That is, when the finger is located in the first area PPG_A1, the processor 140 may extract the PPG signal from the pixel in the first area PPG_A1, and when the finger moves to be located in the second area PPG_A2, the processor 140 may extract the PPG signal from the pixel in the second area PPG_A2. Accordingly, when the finger moves, the PPG signal may be acquired from the same part of the finger.

The PPG signal may be extracted from a single pixel or may be extracted from multiple pixels. When extracting a PPG signal from multi-pixels, the processor 140 may remove a motion component from a pixel value.

For example, an input signal intensity (input intensity: I) may be expressed as a function I (t, x, y) of time t and position (x, y) on a two-dimensional plane, and may be decomposed into an amplitude component and a motion component using a Gaussian distribution as shown in Equation (1) below.

$$I ( t , x , y ) \to A ( t ) \cdot exp [ - \frac{1}{2} \frac{{( x - {x}_{m} ( t ) )}^{2} + {( y - {y}_{m} ( t ) )}^{2}}{{σ}^{2}} ] \qquad ( 1 )$$

In Equation (1), A(t) represents the amplitude component and a component,

$$exp [ - \frac{1}{2} \frac{{( x - {x}_{m} ( t ) )}^{2} + {( y - {y}_{m} ( t ) )}^{2}}{{σ}^{2}} ] ,$$

following the amplitude component represents the motion component. Therefore, when the motion component is removed and only the amplitude component is used, a PPG signal in which motion artifacts have been removed may be acquired.

FIGS. 21A to 21D show noise generated in a PPG signal according to the degree to which a finger moves for a single pixel and multi-pixels. The PPG signal acquired from multi-pixels is a signal acquired from the pixel value in which the motion component has been removed as described above.

Referring to FIG. 21A, when a measurement target (a finger in the present example) of the PPG signal hardly moves (Motion level =0), both the PPG signal acquired from a single pixel and the PPG signal acquired from multi-pixels include almost no noise.

Referring to FIGS. 21B to 21D, it can be seen that as the motion of the measurement target increases, the noise included in the PPG signal acquired from a single pixel also increases. On the other hand, it can be seen that the PPG signal acquired from multi-pixels is not significantly affected by the motion of the measurement target and has a stable shape compared with the PPG signal acquired from a single pixel.

Accordingly, when acquiring the PPG signal, the processor 140 may use the multi-pixels of the front camera 121 to reduce noise appearing in the PPG signal due to the motion of a finger.

Alternatively, whether to use a single pixel or multi-pixels may be determined according to the motion of a finger. The processor 140 may identify the degree to which a finger moves based on at least one of the output of the touch sensor 130 or the output of the front camera 121, and when the degree to which the finger moves is less than a predetermined threshold level, the PPG signal may be acquired from a single pixel. When the degree to which the finger moves is equal to or greater than the predetermined threshold level, the PPG signal may be acquired from multi-pixels.

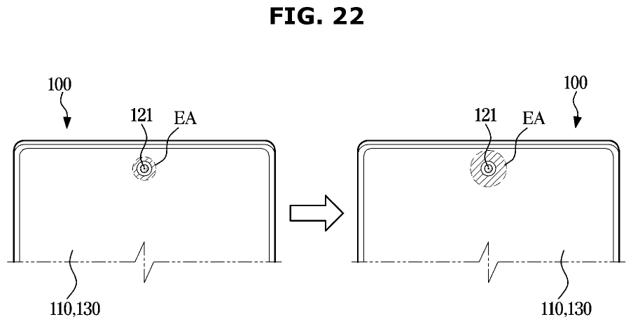

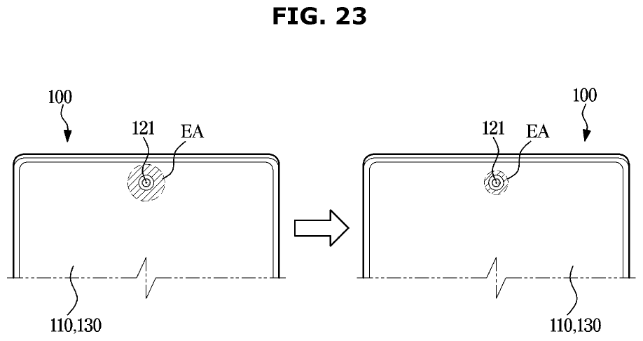

FIGS. 22 and 23 are diagrams illustrating an operation performed by a mobile device to correct distortion caused by a change in contact pressure of a finger according to an embodiment.

Distortion may occur in the PPG signal when the contact pressure of the finger changes during measurement of the PPG signal. In order to prevent distortion due to a change in contact pressure of a finger, the processor 140 may control at least one of a brightness or a size of the emission area EA of the display 110.

The processor 140 may identify a change in the contact pressure of the finger based on at least one of an output of the touch sensor 130 or an output of the front camera 121. A method of identifying the contact pressure is the same as described above.

When it is identified that the finger contact pressure has changed during measurement of the PPG signal, the processor 140 may control at least one of the brightness or the size of the emission area EA of the display 110.

For example, in response to the contact pressure of the finger decreasing during measurement of the PPG signal, the processor 140 may increase the size of the emission area EA of the display 110, or may increase the brightness of the emission area EA of the display 110, as shown in FIG. 22.

Conversely, in response to the contact pressure of the finger increasing during measurement of the PPG signal, the processor 140 may reduce the size of the emission area EA of the display 110 or the brightness of the emission area EA of the display 110, as shown in FIG. 23.

As described above, the size or brightness of the emission area EA of the display 110 may be dynamically changed according to a change in the contact pressure of the finger, such that distortion appearing in the PPG signal may be prevented.

Hereinafter, a method of controlling a mobile device according to an embodiment will be described. In performing the method of controlling the mobile device according to the embodiment, the above-described mobile device 100 may be used. Accordingly, the contents described above with reference to FIGS. 1 to 23 may be equally applied to the method of controlling the mobile device, although not separately mentioned.

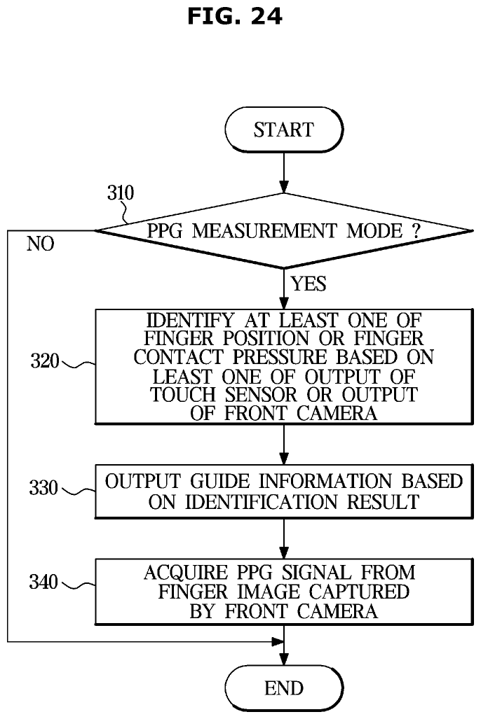

FIG. 24 is a flowchart of a method for controlling a mobile device to provide guide information to a user according to an embodiment.

Referring to FIG. 24, when the mobile device 100 operates in a PPG measurement mode (“Yes” in operation 310), in operation 320, at least one of the position of the finger or the contact pressure of the finger may be identified based on at least one of the output of the touch sensor 130 or the output of the front camera 121.

Guide information for guiding the position of the finger may be output as shown in FIGS. 12 and 13 in response to an application for measuring the PPG signal being executed in the mobile device 100 and biometric information being selected by the user.

When the user's finger is placed on the lens of the front camera 121 according to the output guide information, the touch sensor 130 in an area adjacent to the front camera 121 may come into contact with the user's finger. Accordingly, the processor 140 may identify at least one of the position or the contact pressure of the finger based on the output of the touch sensor 130.

In operation 330, the guide information may be output based on the identification result, and the outputting of the guide information may be achieved using at least one of a visual method, an auditory method, or a tactile method.

In operation 340, when the user's finger is placed at a predetermined position with a predetermined range of pressures, the processor 140 may acquire a PPG signal from a finger image captured by the front camera 121.

In order to acquire the PPG signal, the display 110 may emit light of a specific wavelength from an area (an emission area) corresponding to the front camera 121. The wavelength of light emitted from the emission area EA may be determined based on biometric information to be measured. In this case, light of a single wavelength or multi-wavelengths may be used according to the type of biometric information.

The method of acquiring the PPG signal from the finger image is the same as described above in the embodiment of the mobile device 100.

When the PPG signal is acquired, the processor 140 may acquire biometric information based on the acquired PPG signal, and the acquired biometric information may be provided to the user through the display 110 or the speaker 160.

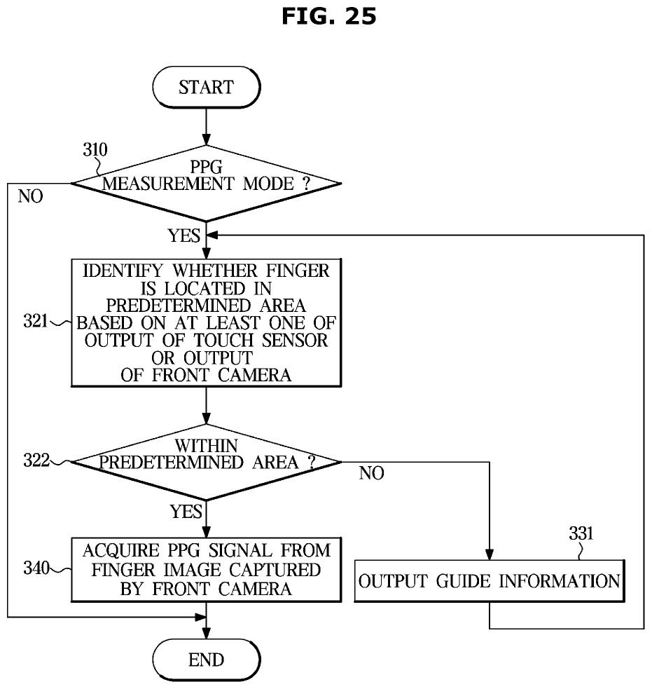

FIG. 25 is a flowchart of a method of controlling a mobile device to output information for guiding the position of a finger according to an embodiment.

Referring to FIG. 25, when the mobile device 100 operates in a PPG measurement mode (“Yes” in operation 310), in operation 321, the processor 140 may identify whether the finger is located in a predetermined area based on at least one of an output of the touch sensor 130 or an output of the front camera 121. The predetermined area may be an area in which a finger needs to be positioned to measure the PPG signal, and may have a predetermined size or a predetermined shape at a predetermined position.

In response to the finger not being located in the predetermined area (“No” in operation 322), in operation 331, the processor 140 may output guide information for guiding the finger to be located in the predetermined area. The guide information may be visually output through the display 110, audibly output through the speaker 160, or tactilely output by generating vibration in the mobile device 100.

The identifying of the position and the outputting of the guide information may be repeatedly performed until the finger is located in the predetermined area, and in response to the finger being located in the predetermined area (“Yes” in operation 322), in operation 340, a PPG signal may be acquired from a finger image captured by the front camera 121 as described above.

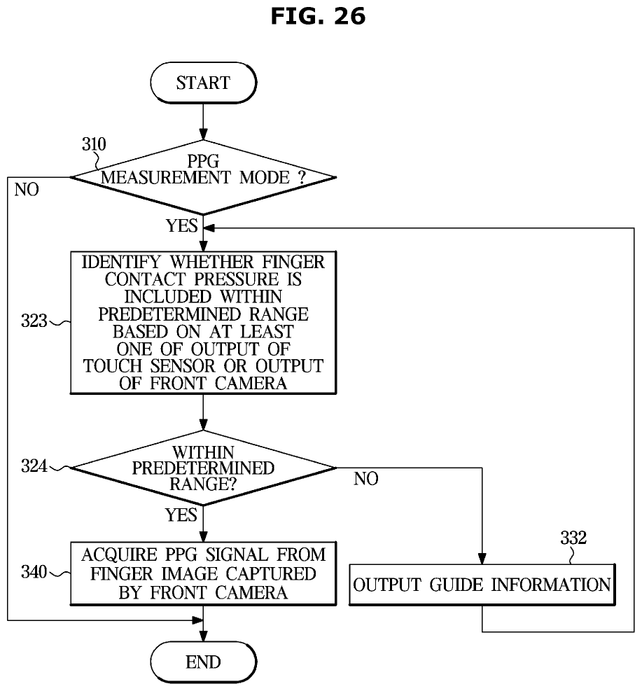

FIG. 26 is a flowchart of a method of controlling a mobile device to output guide information for guiding a contact pressure of a finger according to an embodiment.

Referring to FIG. 26, when the mobile device 100 operates in a PPG measurement mode (“Yes” in operation 310), in operation 323, the processor 140 may identify whether the finger contact pressure falls within a predetermined range based on at least one of an output of the touch sensor 130 or an output of the front camera 121. The predetermined range for the contact pressure may be a range of pressures suitable for acquiring a PPG signal, and the pressure may be set and stored through a test performed when the PPG measurement mode is first executed. Alternatively, the suitable pressure may also be set and stored in advance by experiments, simulations, theories, statistics, etc., in the manufacturing stage of the mobile device 100.

In response to the finger contact pressure not being included in the predetermined range (“No” in operation 324), in operation 332, the processor 140 may output guide information for guiding the finger contact pressure to fall within the predetermined range. The guide information may be visually output through the display 110, audibly output through the speaker 160, or tactilely output by generating vibrations in the mobile device 100.

The identifying of the contact pressure and the outputting of the guide information may be repeatedly performed until the finger contact pressure falls within the predetermined range, and in response to the finger contact pressure being within the predetermined range (“Yes” in operation 324), in operation 340, a PPG signal may be acquired from the finger image captured by the front camera 121 as described above.

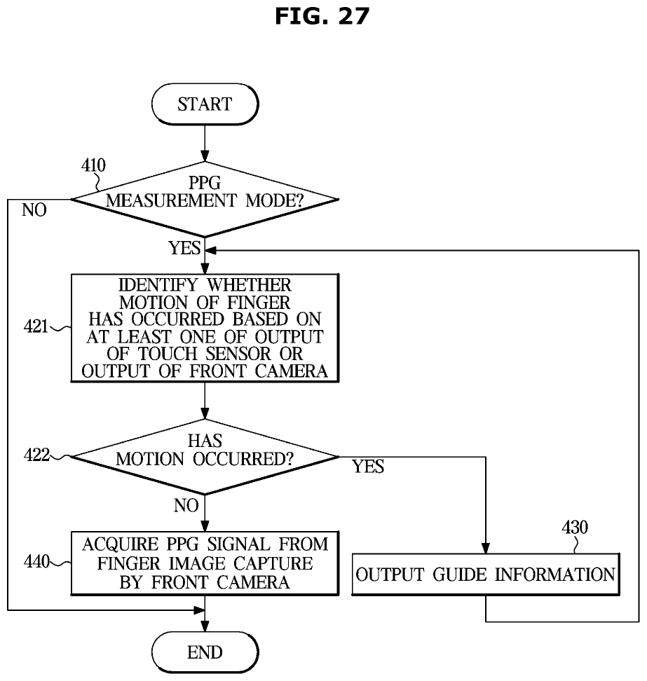

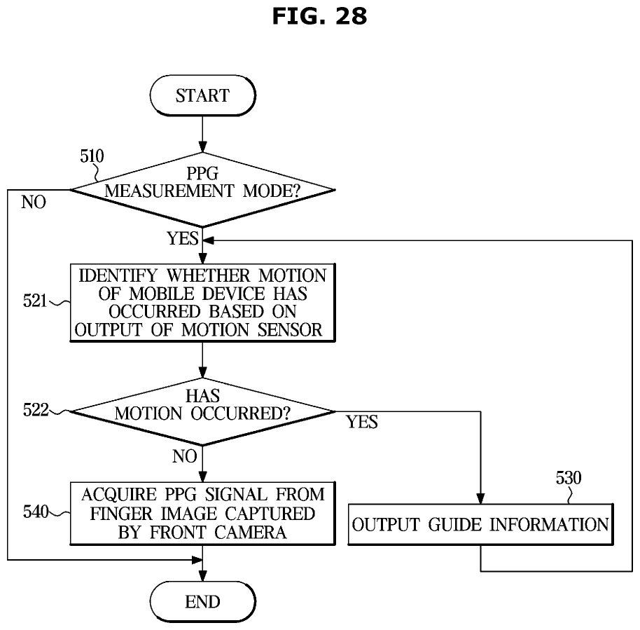

FIG. 27 is a flowchart of a method of controlling a mobile device to output guide information with regard to a motion of a finger according to an embodiment. FIG. 28 is a method of controlling a mobile device to output guide information with regard to a motion of a mobile device according to an embodiment..

When the finger moves during measurement of the PPG signal, noise or artifacts due to the motion may occur. Accordingly, the method of controlling a mobile device according to an embodiment may guide the user not to move while measuring the PPG signal.

Referring to FIG. 27, when the mobile device 100 operates in a PPG measurement mode (“Yes” in operation 410), in operation 421, the processor 140 may identify whether a motion of the finger has occurred based on at least one of the output of the touch sensor 130 or the output of the front camera.

In response to identifying that a motion has occurred (“Yes” in operation 422), in operation 430, guide information for indicating that a motion is not allowed may be output. The guide information may be visually output through the display 110, audibly output through the speaker 160, or tactilely output by generating vibration in the mobile device 100.

In response to no motion having occurred (“No” in operation 422), in operation 440, the processor 140 may acquire a PPG signal from the finger image captured by the front camera.

When a motion of the mobile device 100 has occurred during measurement of the PPG signal, distortion may occur in the PPG signal. Referring to FIG. 28, when the mobile device 100 operates in a PPG measurement mode (“Yes” in operation 510), in operation 521, the processor 140 may identify whether a motion of the mobile device 100 has occurred based on the output of the motion sensor 170 provided in the mobile device 100.

In response to identifying that a motion has occurred (“Yes” in operation 522), in operation 530, guide information indicating that a motion is not allowed may be output. The guide information may be visually output through the display 110, audibly output through the speaker 160, or tactilely output by generating vibration in the mobile device 100.

In response to no motion having occurred (“No” in operation 522), in operation 540, the processor 140 may acquire a PPG signal from the finger image captured by the front camera.

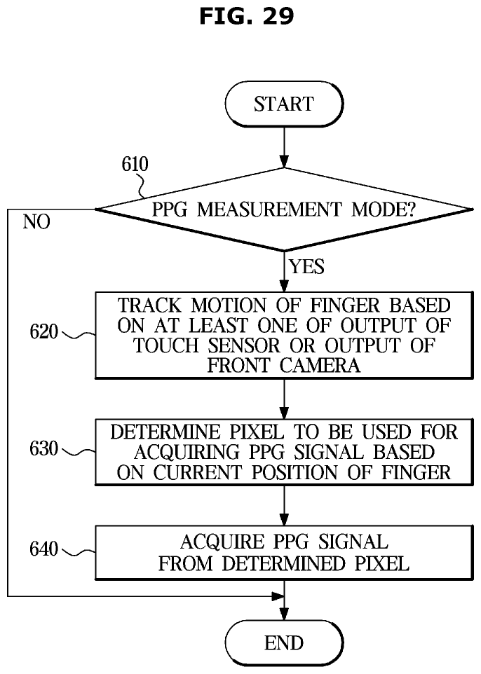

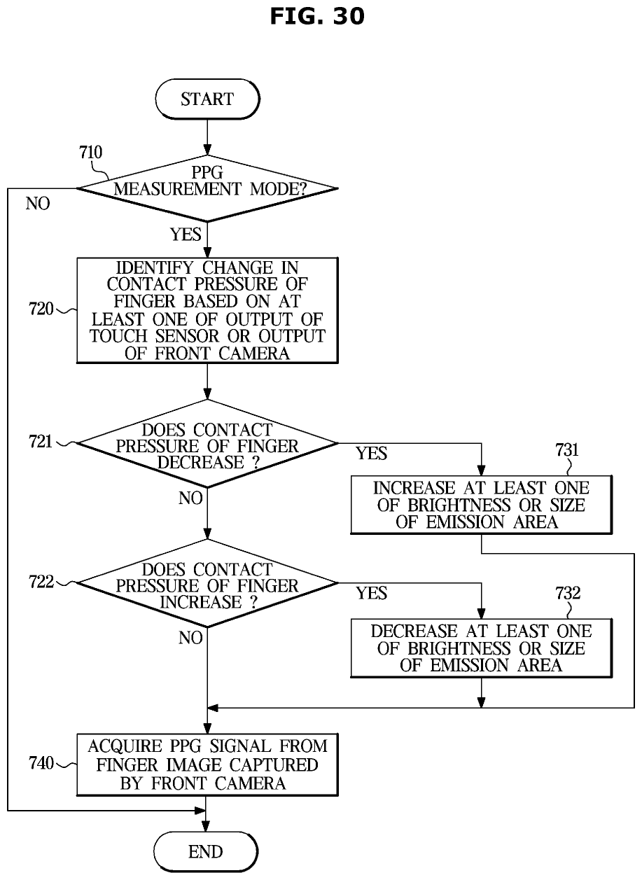

In the method of controlling a mobile device according to an embodiment, when the user moves during measurement of the PPG signal, the process of measuring the PPG signal may be controlled to prevent distortion of the PPG signal. Hereinafter, the control of the measurement process of the PPG signal will be described with reference to FIGS. 29 and 30.

FIG. 29 is a flowchart a method of controlling a mobile device to prevent distortion due to a change in position of a finger according to an embodiment. FIG. 30 is a flowchart a method of controlling a mobile device to prevent distortion due to a change in contact of a finger according to an embodiment.

Referring to FIG. 29, when the mobile device 100 operates in a PPG measurement mode (“Yes” in operation 610), in operation 620, a motion of the finger may be tracked based on at least one of an output of a touch sensor or an output of the front camera.

In operation 630, the processor 140 may determine a pixel to be used for acquiring a PPG signal based on the current position of the finger, and, in operation 640, the processor 140 may acquire a PPG signal from the determined pixel. As described above, the acquiring of the PPG signal may include using a plurality of frame images captured by the front camera 121, in which the plurality of frame images may be captured according to a set frame rate and transmitted to the processor 140. The processor 140 may extract the PPG signal from at least one pixel corresponding to the current position of the finger in the transmitted frame image. That is, as described above with reference to FIG. 20, when the finger is located in the first area PPG_A1, the PPG signal is extracted from the pixel in the first area PPG_A1, and when the finger moves to be located in the second area PPG_A2, the PPG signal may be extracted from the pixel of the second area PPG_A2. Accordingly, even when the finger moves, the PPG signal may be acquired from the same part of the finger.

Referring to FIG. 30, when the mobile device 100 operates in a PPG measurement mode (“Yes” in operation 710), in operation 720, the processor 140 may identify a change in the contact pressure of the finger based on at least one of an output of the touch sensor 130 or an output of the front camera 121.

In response to the contact pressure of the finger decreasing during measurement of the PPG signal (“Yes” in operation 721), in operation 731, the processor 140 may increase at least one of the brightness or size of the emission area EA.

In response to the contact pressure of the finger increasing during measurement of the PPG signal (“No” in operation 721, and “Yes” in operation 722), in operation 732, the processor 140 may decrease at least one of the brightness or size of the emission area EA.

In response to no change in the contact pressure (“No” in operation 722) or in response to at least one of the brightness or size of the emission area EA being adjusted according to the change in the contact pressure, in operation 740, the processor 140 may acquire a PPG signal from a finger image captured by the front camera.

According to the examples of FIGS. 29 and 30 described above, even when a finger moves during measurement of the PPG signal, distortion is prevented from occurring in the PPG signal by changing the pixel from which the PPG signal is to be extracted or adjusting the size or brightness of the emission area EA.

On the other hand, the computer program according to an embodiment may be a computer program that is stored in a recording medium to perform the operations described in the embodiment of the operations of the processor 140 and the method of controlling the mobile device described in the embodiment of the mobile device 100 described above in combination with the mobile device 100. The computer program may be installed by default in the mobile device 100 as described above, or may be installed by a user after the mobile device 100 is sold.

Since all operations executed by the computer program according to the embodiment are the same as those of the descriptions in the embodiment of the mobile device 100 and the embodiment of the method of controlling the mobile device, descriptions thereof will be omitted herein.

According to the mobile device, the control method thereof, and the computer program stored in the recording medium in combination with the mobile device described above, a PPG signal may be measured using components provided in the mobile device, such as a display, a front camera, and a touch sensor, without adding additional equipment.

In addition, the position or contact pressure of an object may be identified based on the output of the front camera or the output of the touch sensor, and suitable guide information may be output based on the identification result, such that accurate measurement of the PPG signal may be allowed.

In addition, the user's motion may be tracked based on the output of the front camera or the output of touch sensor, the pixel from which the PPG signal is to be extracted may be changed or the size or brightness of the emission area may be adjusted such that distortion is prevented from occurring in the PPG signal.

As disclosed herein, a mobile device, a method of controlling the same, and a computer program stored in a recording medium may measure a PPG signal using a display and a camera provided in the mobile device to thereby measure a PPG signal without having additional components or equipment and acquire biometric information based on the PPG signal.

As disclosed herein, a mobile device, a method of controlling the same, and a computer program stored in a recording medium may provide guide information to a user or correct distortion due to a motion based on a position or contact pressure of a finger identified using a touch sensor or a front camera provided in the mobile device to thereby improve the accuracy and reliability of a PPG signal using a basic configuration provided in the mobile device without having additional components or equipment.

The foregoing detailed descriptions may be merely an example of the disclosure and the aspects disclosed herein may be used through various combinations, modifications and environments. The aspects disclosed herein may be amended or modified, not being out of the scope, technical idea or knowledge in the art. Further, it is not intended that the scope of this application be limited to these specific embodiments or to their specific features or benefits. Rather, it is intended that the scope of this application be limited solely to the claims which now follow and to their equivalents. Further, the appended claims should be appreciated as a step including even another embodiment.

What is claimed is:

1. A mobile device comprising:
a display;
a front camera provided to face in a forward direction of the display;
a touch sensor provided at a front side of the display; and
a processor configured to:
acquire a photoplethysmography (PPG) signal from an image of a finger captured by the front camera in a PPG measurement mode, and
output guide information that guides at least one of a position of the finger or a contact pressure with which the finger presses the front camera based on at least one of an output of the touch sensor or the image of the finger.

2. The mobile device of claim 1, wherein the display is configured to emit light of a specific wavelength in an area corresponding to the front camera in the PPG measurement mode, and
wherein the front camera is configured to, upon the light emitted from the display in the PPG measurement mode being reflected from or transmitted through the finger, receive the light reflected from or transmitted through the finger thereby capturing the image of the finger.

3. The mobile device of claim 2, wherein the processor is further configured to:
identify whether the finger is located in a predetermined area on the display based on the output of the touch sensor, and
output the guide information with at least one of a visual method, an auditory method, or a tactile method based on a result of the identification.

4. The mobile device of claim 3, wherein the processor is further configured to control the display to display a guide image that guides the position of the finger to the predetermined area.

5. The mobile device of claim 2, wherein the processor is further configured to:
identify whether the contact pressure is included within a predetermined range based on the output of the touch sensor, and
output the guide information with at least one of a visual method, an auditory method, or a tactile method based on a result of the identification.

6. The mobile device of claim 5, further comprising a speaker,
wherein the processor is further configured to control the speaker to present a guide speech that guides the contact pressure to be provided within the predetermined range.

7. The mobile device of claim 2, wherein the processor is further configured to:
identify whether a motion of the finger occurs based on the at least one of the output of the touch sensor or the image of the finger, and
based on identifying that the motion of the finger occurs, output the guide information with at least one of a visual method, an auditory method, or a tactile method.

8. The mobile device of claim 2, wherein the processor is further configured to, based on at least one of the output of the touch sensor or the image of the finger, perform a distortion preventive process that prevents distortion due to a motion of the finger.

9. The mobile device of claim 8, wherein the processor is further configured to:
track the motion of the finger based on at least one of the output of the touch sensor or the image of the finger, and
determine, based on a current position of the finger, at least one pixel from among a plurality of pixels of the image of the finger to use to acquire the PPG signal.

10. The mobile device of claim 8, wherein the processor is further configured to:
identify a degree to which the finger has moved based on at least one of the output of the touch sensor or the image of the finger, and
determine, based on the degree to which the finger has moved, to use a single pixel or multiple pixels to acquire the PPG signal.

11. The mobile device of claim 2, further comprising a motion sensor configured to detect a motion of the mobile device,
wherein the processor is further configured to output guide information related to the motion of the mobile device with at least one of a visual method, an auditory method, or a tactile method, based on an output of the motion sensor.

12. The mobile device of claim 2, wherein the processor is further configured to
identify a change in the contact pressure based on at least one of the output of the touch sensor or the image of the finger, and
control at least one of a size or a brightness of a light emission area in which the light of the specific wavelength is emitted, based on the change in the contact pressure.

13. The mobile device of claim 2, wherein the processor is further configured to control the display to alternately emit light rays of a plurality of different specific wavelengths in the PPG measurement mode.

14. The mobile device of claim 2, wherein the processor is further configured to acquire biometric information of a user based on the acquired PPG signal, and
wherein the biometric information of the user includes at least one of a heart rate, a blood oxygenation, a stress index, a respiration rate, a blood pressure, an oxygen delivery time, or a pulse speed.

15. A method of controlling a mobile device including a display, a front camera provided to face in a forward direction of the display, and a touch sensor provided at a front side of the display, the method comprising:
identifying at least one of a position of a finger or a contact pressure with which the finger presses the front camera based on at least one of an output of the touch sensor or an image of the finger captured by the front camera;
outputting guide information that guides at least one of the position of the finger or the contact pressure based on a result of the identifying step; and
acquiring a photoplethysmography (PPG) signal from the image of the finger captured by the front camera.

16. The method of claim 15, wherein the identifying at least one of the position of the finger or the contact pressure includes identifying whether the finger is located in a predetermined area on the display based on the output of the touch sensor, and
wherein the outputting of the guide information includes outputting the guide information with at least one of a visual method, an auditory method, or a tactile method based on a result of the identifying whether the finger is located in a predetermined area on the display .

17. The method of claim 15, wherein the identifying of at least one of the position of the finger or the contact pressure includes:
identifying whether the contact pressure is included within a predetermined range based on the output the touch sensor, and
the outputting of the guide information includes outputting the guide information using at least one of a visual method, an auditory method, or a tactile method based on a result of the identification.

18. The method of claim 15, wherein the identifying of the at least one of the position of the finger or the contact pressure includes identifying whether a motion of the finger occurs based on at least one of the output of the touch sensor or the image of the finger, and
wherein the outputting of the guide information includes, in response to identifying that the motion of the finger has occurred, outputting the guide information with at least one of a visual method, an auditory method, or a tactile method.

19. The method of claim 15, further comprising performing a distortion preventive process that prevents distortion due to a motion of the finger based on at least one of the output of the touch sensor or the image of the finger.

20. The method of claim 19, wherein the performing of the distortion preventive process includes:
tracking the motion of the finger based on at least one of the output of the touch sensor or the image of the finger, and determining based on a current position of the finger, at least one pixel from among a plurality of pixels of the image of the finger to use to acquire the PPG signal ; or
identifying a change in the contact pressure based on at least one of the output of the touch sensor or the image of the finger, and controlling at least one of a size or a brightness of a light emission area in the display, in which light of a specific wavelength is emitted, based on the change in the contact pressure.
